(19) United States
(12) Patent Application Publication
(10) Pub. No.: US 2014/0046135 A1
(43) Pub. Date: Feb. 13, 2014

(54) MEASUREMENT PROBE

(71) Applicant: OLYMPUS MEDICAL SYSTEMS CORP., Tokyo (JP)

(72) Inventor: Kazuhiro Gono, Sagamihara-shi (JP)

(21) Appl. No.: 13/917,322

(22) Filed: Jun. 13, 2013

Related U.S. Application Data
(60) Provisional application No. 61/681,358, filed on Aug. 9, 2012.

(51) Int. Cl. A61B 1/07

(52) U.S. Cl. CPC A61B 1/07
USPC 600/175

(57) ABSTRACT

A measurement probe is detachably connected to a living body optical measurement apparatus which optically measures a body tissue. The measurement probe includes an illumination fiber which radiates illumination light on the body tissue, a light reception fiber which receives return light of the illumination light reflected and/or scattered by the body tissue, an optical element which abuts on the illumination fiber and the light reception fiber, and fixes a distance between the illumination fiber and the light reception fiber, and the body tissue, an optical member which is provided at a tip of the measurement probe, and which includes a hollow portion which forms a hollow space, and a transmittance change unit which is provided in the hollow portion, and which changes a transmittance of light incident on an interior of the measurement probe in response to a load from an outside.

### CROSS-REFERENCE TO RELATED APPLICATIONS

This application is based upon and claims the benefit of priority from U.S. Provisional Patent Application No. 61/681,358, filed on Aug. 9, 2012, the entire contents of which are incorporated herein by reference.

### BACKGROUND OF THE INVENTION

1. Field of the Invention

The disclosure relates to a measurement probe which is connected to an optical measurement apparatus which measures optical characteristics of body tissues.

2. Description of the Related Art

Conventionally, an optical measurement system is known which radiates illumination light on a sample such as body tissues, and estimates the property of the sample based on a measurement value of detection light reflected or scattered from the sample. This optical measurement system is formed with an optical measurement apparatus which has a light source which emits illumination light to a sample and a detection unit which detects detection light from the sample, and a measurement probe which is detachable from this optical measurement apparatus, and which radiates illumination light on the sample and receives light from the sample (see, for example, Japanese Patent Application Laid-open No. 2002-291764 and Japanese Patent Application Laid-open No. 2006-158716). The measurement probe radiates illumination light on body tissues from a tip of an illumination fiber connected to the light source, receives light emitted from the body tissues as a result of this radiation using a plurality of light reception fibers, and measures a light intensity distribution.

Meanwhile, for example, at the tip of the measurement probe introduced in a subject, an optical member which seals the measurement probe is provided. In the measurement probe, the illumination light radiated from the tip of the illumination fiber passes through this optical element, and the light reflected from the body tissues transmits through the optical member and is incident on the light reception fibers.

### SUMMARY OF THE INVENTION

In some embodiments, a measurement probe is detachably connected to a living body optical measurement apparatus which optically measures a body tissue, and includes: an illumination fiber which radiates illumination light on the body tissue; a light reception fiber which receives return light of the illumination light reflected and/or scattered by the body tissue; an optical element which abuts on the illumination fiber and the light reception fiber, and fixes a distance between the illumination fiber and the light reception fiber, and the body tissue; an optical member which is provided at a tip of the measurement probe, and which includes a hollow portion which forms a hollow space; and a transmittance change unit which is provided in the hollow portion, and which changes a transmittance of light incident on an interior of the measurement probe in response to a load from an outside.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram schematically illustrating a configuration of a living body optical measurement system according to an embodiment of the present invention.

FIG. 2 is a view schematically illustrating a cross section obtained by cutting along a longitudinal direction a tip portion of a measurement probe including an optical element of the living body optical measurement system according to an embodiment of the present invention.

FIG. 3 is a plan view schematically illustrating the measurement probe in an A direction from an arrow view in FIG. 2.

FIG. 4 is a view illustrating a situation in which the living body optical measurement system according to an embodiment of the present invention is used in an endoscope system.

FIG. 5 is a plan view schematically illustrating the measurement probe in the A direction from an arrow view in FIG. 2.

FIG. 6 is a view schematically illustrating a cross section obtained by cutting along the longitudinal direction a tip portion of a measurement probe according to Modified Example 1 of the embodiment of the present invention.

FIG. 7 is a view schematically illustrating a cross section obtained by cutting along the longitudinal direction a tip portion of a measurement probe according to Modified Example 2 of the embodiment of the present invention.

### DETAILED DESCRIPTION OF THE PREFERRED EMBODIMENTS

Hereinafter, a preferred embodiment of a measurement probe according to the present invention will be described in detail with reference to the drawings. Incidentally, this embodiment by no means limits the present invention. Further, the same portions will be described by assigning the same reference numerals in the drawings. Furthermore, it needs to be kept in mind that the drawings are schematic, and the relationship between the thickness and the width of each member and the ratio of each member are different from actual members. Still further, there are portions including a different relationship between dimensions and a different ratio even between the drawings.

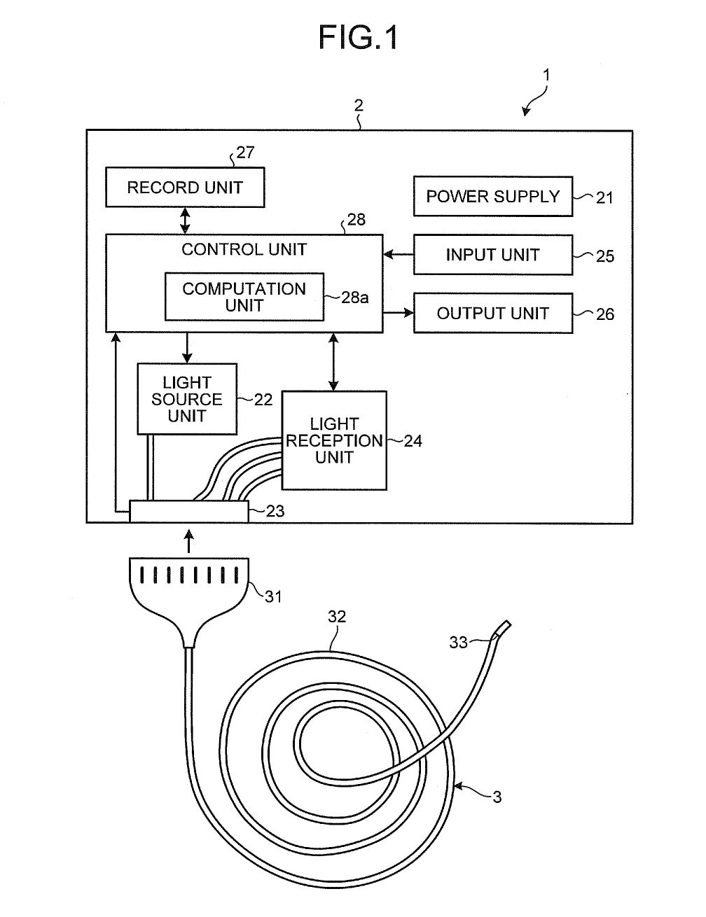

FIG. 1 is a block diagram schematically illustrating a configuration of a living body optical measurement system according to an embodiment of the present invention. A living body optical measurement system 1 illustrated in FIG. 1 has a living body optical measurement apparatus 2 which optically measures a measurement target such as body tissues which are scatters and detects the property of the measurement target (characteristics), and a measurement probe 3 which is used for measurement and which is detachable from the living body optical measurement apparatus 2 and is inserted in a subject.

First, the living body optical measurement apparatus 2 will be described. The living body optical measurement apparatus 2 has a power supply 21, a light source unit 22, a connection unit 23, a light reception unit 24, an input unit 25, an output unit 26, a record unit 27 and a control unit 28. The power supply 21 supplies power to each component of the living body optical measurement apparatus 2.

The light source unit 22 is realized by using, for example, an incoherence light source such as a white LED (Light Emitting Diode), a xenon lamp, a tungsten lamp or a halogen lamp, and one or a plurality of lenses where necessary such as condenser lenses or collimator lenses. The light source unit 23 outputs incoherent light which includes at least one spectrum component and which is radiated on a measurement target through the connection unit 23, to the measurement probe 3.

The connection unit 23 detachably connects a connector portion 31 of the measurement probe 3 to the living body optical measurement apparatus 2. The connection unit 23 outputs light emitted from the light source unit 22, to the measurement probe 3, and outputs return light of illumination light which is emitted from the measurement probe 3 and which is reflected and/or scattered by a measurement target, to the light reception unit 24. The connection unit 23 outputs information as to whether or not the measurement probe 3 is connected, to the control unit 28.

The light reception unit 24 receives and measures return light of illumination light which is emitted from the measurement probe 3 and which is reflected and/or scattered by a measurement target. The light reception unit 24 is realized by using, for example, a plurality of spectrometers and light reception sensors. More specifically, in the light reception unit 24, the number of spectrometers corresponds to the number of light reception fibers of the measurement probe 3 which will be described below. The light reception unit 24 measures a spectrum component and an intensity distribution of scattering light incident from the measurement probe 3, and measures each wavelength. The light reception unit 24 outputs a measurement result to the control unit 28.

The input unit 25 is realized by using, for example, a push-type switch or a touch panel, and receives an input of an instruction signal for instructing activation of the living body optical measurement apparatus 2 or an instruction signal for instructing other various operations and outputs the instruction signal to the control unit 28.

The output unit 26 is realized by using, for example, a liquid crystal or organic EL (Electro Luminescence) display and speakers, and outputs information related to various processing in the living body optical measurement apparatus 2. Further, the output unit 26 displays a numerical value such as an intensity of light received by the light reception unit 24 (a characteristic value computed by a computation unit 28a described below), on a display under control of the control unit 28.

The record unit 27 is realized by using volatile memory or non-volatile memory, and records various programs for operating the living body optical measurement apparatus 2, and various items of data and various parameters used for optical measurement processing. The record unit 27 temporarily records information which is obtained during processing in the living body optical measurement apparatus 2. Further, the record unit 27 associates and records a measurement result of the body living optical measurement apparatus 2 and a subject of the measurement target. Incidentally, the record unit 27 may be configured by using, for example, a memory card attached from an outside of the living body optical measurement apparatus 2.

The control unit 28 is configured by using, for example, a CPU (Central Processing Unit). The control unit 28 controls a processing operation of each unit of the living body optical measurement apparatus 2. The control unit 28 controls the operation of the living body optical measurement apparatus 2 by, for example, transferring instruction information matching each unit of the living body optical measurement apparatus 2 or data. The control unit 28 records the measurement result of the light reception unit 24, in the record unit 27. The control unit 28 has the computation unit 28a.

The computation unit 28a performs a plurality of computation processings based on the measurement result of the light reception unit 24, and computes a characteristic value related to the property of a measurement target. The type of this characteristic value is set according to, for example, an instruction signal received by the input unit 25.

Next, the measurement probe 3 will be described. The measurement probe 3 is realized by disposing a plurality of optical fibers inside. More specifically, the measurement probe 3 is realized by using an illumination fiber which emits illumination light to a measurement target, and a plurality of light reception fibers on which return light of illumination light reflected and/or scattered by the measurement target is incident at different angles. The measurement probe 3 has a connector portion 31 which is detachably connected to the connection unit 23 of the living body optical measurement apparatus 2, a flexible portion 32 which has flexibility, and a tip portion 33 which radiates illumination light supplied from the light source unit 22 and receives return light from a measurement target.

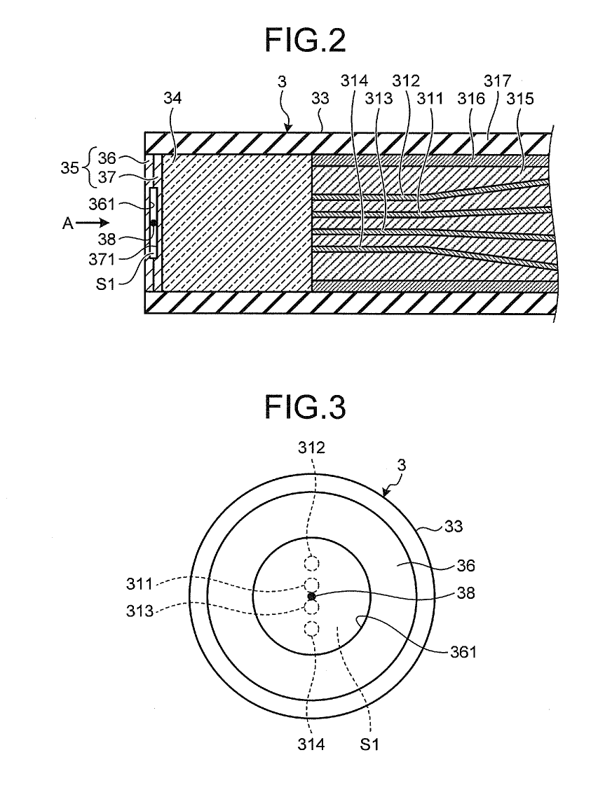

A configuration of the tip portion 33 of the measurement probe 3 will be described in detail. FIG. 2 is a view schematically illustrating a cross section obtained by cutting along a longitudinal direction the tip portion 33 of the measurement probe 3. FIG. 3 is a plan view schematically illustrating the measurement probe 3 in an A direction from an arrow view in FIG. 2. As illustrated in FIG. 2, the tip portion 33 is provided with an optical element 34 and a glass window 35 (optical member) which is provided at the tip and which forms part of an outer surface of the measurement probe 3.

The measurement probe 3 has: an illumination fiber 311 which radiates illumination light on a measurement target; a first light reception fiber 312 (first light reception channel), a second light reception fiber 313 (second light reception channel) and a third light reception fiber 314 (third light reception channel) on which return light of the illumination light reflected and/or scattered by the measurement target is incident; a covering member 315, such as glass or resin, which prevents damages on and fixes positions of the illumination fiber 311, the first light reception fiber 312, the second light reception fiber 313 and the third light reception fiber 314; a protection portion 316, such as glass or brass, which protects the covering member 315 from an external force; and a probe outer casing 317 which is made of, for example, SUS, and which covers an outer peripheral surface of the protection portion 316, the optical element 34 and the glass window 35.

The illumination fiber 311 is formed by using a step-index single core fiber. The illumination fiber 311 transmits illumination light outputted from the light source unit 22, and radiates the illumination light on a measurement target through the optical element 34. Incidentally, the number of illumination fibers 311 can be adequately changed according to an examination item or a type of a measurement target such as a blood current or a site.

The first light reception fiber 312, the second light reception fiber 313 and the third light reception fiber 314 are each formed by using a step-index single core fiber. The first light reception fiber 312, the second light reception fiber 313 and the third light reception fiber 314 each transmit return light of illumination light which is incident on each tip through the optical element 34 and which is reflected and/or scattered by a measurement target, and outputs the light to the light reception unit 24 of the living body optical measurement apparatus 2. Incidentally, the number of light reception fibers can be adequately changed according to an examination item or a type of a measurement target such as a blood current or a site.

The optical element 34 has a columnar shape, and is formed by using a transmissive glass having a predetermined refractive index. The optical element 34 is formed to fix the distance between the illumination fiber 311 and a measurement target, and radiate light in a state where a spatial coherent length is fixed. Further, the optical element 34 is formed to fix the distance between the first light reception fiber 312 and the measurement target, the distance between the second light reception fiber 313 and the measurement target and the distance between the third light reception fiber 314 and the measurement target, respectively, and stably receive return light at a predetermined scattering angle. Meanwhile, the distance between each fiber and the measurement target refers to a distance in a direction of a center axis of the column of the optical element 34 and refers to the distance from the tip of the tip portion 33 to each fiber, and, preferably, is determined taking the thickness of the glass window 35 into account. Incidentally, if the thickness of the glass window 35 can be neglected upon comparison with the distance of the optical element 34, this does not apply when this distance is determined.

The glass window 35 has a virtually circular disk shape, and is formed by pasting glass members 36 and 37 formed by using a transmissive glass having a predetermined refractive index. The glass members 36 and 37 are formed to stably receive return light at a predetermined scattering angle similar to the optical element 34. Further, the surface of the measurement target is flattened at the end surface of the glass window 35 (glass member 36), so that it is possible to measure the measurement target without an influence of a concave-convex shape of the surface of the measurement target.

Furthermore, in the glass members 36 and 37, concave portions 361 and 371 having concave shapes are formed on the surface side on which the glass members 36 and 37 face each other when pasted. The concave portions 361 and 371 have the same shape, and form a hollow portion S1 which is a circular disk-shaped hollow space by pasting the glass members 36 and 37 with respective outer rims aligned.

In the hollow portion S1 formed by the concave portions 361 and 371, a sealing member 38 which seals a liquid inside is provided. The sealing member 38 is made of resin having such quality of a material or the thickness that the sealing member 38 is broken by, for example, a predetermined load. For a liquid to be sealed inside the sealing member 38, a pigment or a material such as a black ink which changes the transmittance of light incident on the interior of the tip portion 33 is used.

Further, the sealing member 38 is disposed at a position which does not interfere with at least an axis of the illumination fiber 311 extending in parallel to the longitudinal direction of the tip portion 33 (an optical axis of illumination light emitted from the illumination fiber 311) and axes of the first light reception fiber 312, the second light reception fiber 313 and the third light reception fiber 314 in parallel to the longitudinal direction of the tip portion 33 (the optical axes of light (return light) incident on each light reception fiber).

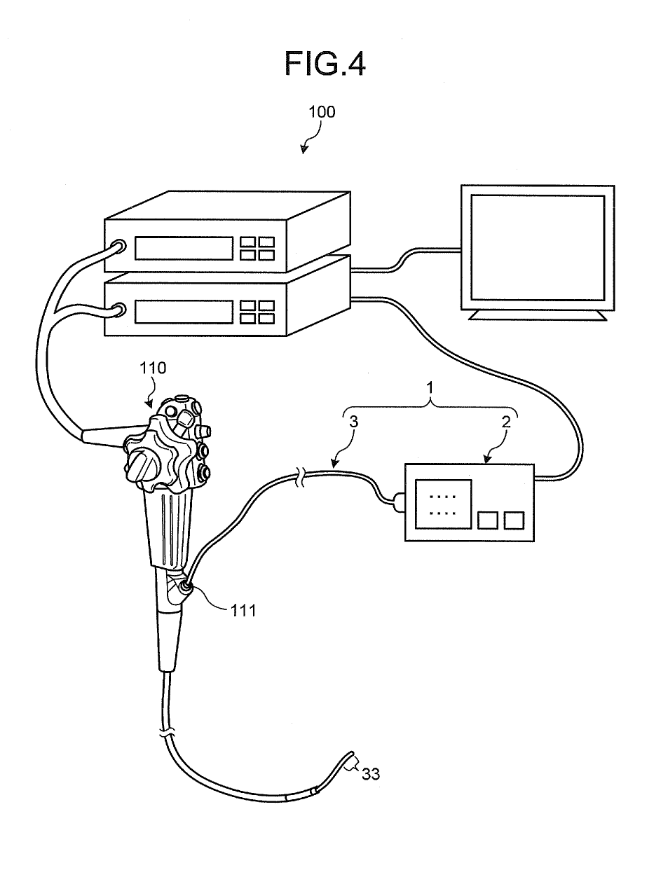

As illustrated in FIG. 4, with the living body optical measurement system 1 configured as described above, the measurement probe 3 is inserted in a subject through the treatment tool channel 111 provided in an endoscope apparatus 110 (endoscope) of the endoscope system 100, the illumination fiber 311 radiates illumination light on a measurement target, and the first light reception fiber 312, the second light reception fiber 313 and the third light reception fiber 314 each receive return light of illumination light reflected and/or scattered by the measurement target, at different scattering angles and transmit the light to the light reception unit 24 of the living body optical measurement apparatus 2. Then, the computation unit 28a computes a characteristic value of the property of the measurement target based on the measurement result of the light reception unit 24.

Meanwhile, when an external load is applied to the glass window 35 (glass member 36), the concave portion 361 is deformed, and the volume of the hollow portion S1 formed by the concave portions 361 and 371 changes. Following this change in the volume, the load is also applied to the sealing member 38 to be deformed. In this case, when a load of a predetermined magnitude or more is applied to the glass window 35 (glass member 36), and this load deforms the sealing member 38, the sealing member 38 is broken. Incidentally, with the present embodiment, the load of a predetermined magnitude is greater than a load applied upon contact with an inner wall of a body cavity in a subject, and is a load equal to or less than the strength of the load at which the glass window 35 (glass member 36) is broken. That is, the load is the magnitude of a force produced which damages the glass window 35 upon, for example, contact.

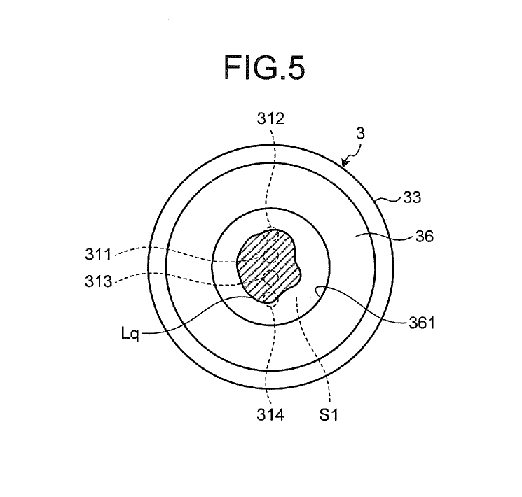

FIG. 5 is a plan view schematically illustrating the measurement probe in the A direction from an arrow view in FIG. 2, and is a view illustrating that a load of a predetermined magnitude or more is applied to the glass window 35. When the sealing member 38 is broken due to the external load, a liquid Lq inside the sealing member 38 is released inside the hollow portion S1. In this case, the liquid Lq spreads covering the position interfering with optical axes of light (return light) incident on at least the first light reception fiber 312, the second light reception fiber 313 and the third light reception fiber 314.

By this means, the transmittance of light incident on the first light reception fiber 312, the second light reception fiber 313 and the third light reception fiber 314 decreases. That is, the intensity (numerical value) of light measured by the measurement probe 3 remarkably decreases, and optical performance of the measurement probe 3 deteriorates compared to initial performance adjusted upon shipping.

Further, when a black ink is used for the liquid Lq, the interior of the glass window 35 is stained with the black ink, so that it is possible to visually check that the optical performance of the measurement probe 3 deteriorates compared to the initial performance.

According to the above-described embodiment of the present invention, the sealing member 38 is disposed in the hollow portion S1 inside the glass window 35, when a load of a predetermined magnitude or more is applied, the sealing member 38 is broken, and the liquid Lq sealed inside the sealing member 38 is released inside the hollow portion S1 covering at least the first light reception fiber 312, the second light reception fiber 313 and the third light reception fiber 314, and the transmittance is changed, so that it is possible to check whether or not optical performance of the measurement probe 3 is maintained to initial performance.

Further, according to an embodiment of the present invention, by checking the interior of the glass window 35 by using a black ink for the liquid Lq, it is possible to visually decide whether or not optical performance of the measurement probe 3 deteriorates compared to initial performance.

Furthermore, according to an embodiment of the present invention, the measurement probe 3 is detachable from the living body optical measurement apparatus 2, so that the measurement probe 3 is disposable, the measurement probe 3 does not need to be sterilized at medical facilities, and the measurement probe 3 may have comparatively poor durability and, consequently, it is possible to reduce cost of the measurement probe 3.

In addition, with the above embodiment, when seen from a direction orthogonal to the bottom surface, the concave portions 361 and 371 may have, for example, any outer rim shape as long as the illumination fiber 311, the first light reception fiber 312, the second light reception fiber 313 and the third light reception fiber 314 are included in the bottom surface. Further, when the glass members 36 and 37 are pasted, if the hollow portion S1 formed by the concave portions 361 and 371 is sealed, the outer rim shapes of the concave portions 361 and 371 may not match, and may have different shapes and form a hollow space having a step shape.

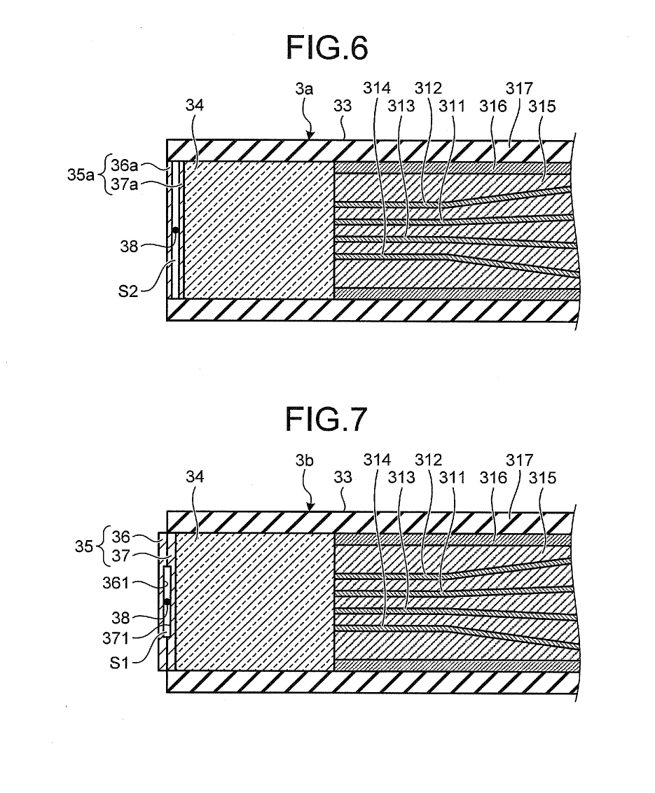

FIG. 6 is a view schematically illustrating a cross section obtained by cutting along the longitudinal direction the tip portion 33 of a measurement probe 3a according to Modified Example 1 of the embodiment of the present invention. According to Modified Example 1, a glass window 35a is provided instead of a glass window 35 according to the above embodiment. The other configurations are the same as in the above embodiment.

The glass window 35a has a virtually circular disk shape, and is provided with flat glass members 36a and 37a which are formed by using transmissive glasses having a predetermined refractive index and which are spaced apart. The glass members 36a and 37a are formed to stably receive return light at a predetermined scattering angle similar to the optical element 34. Further, the surface of a measurement target is flattened at the end surface of the glass window 35a (glass member 36a), so that it is possible to measure the measurement target without an influence of a concave-convex shape of the surface of the measurement target.

Further, the glass members 36a and 37a are disposed in the tip portion 33 while being spaced apart from each other, and the glass members 36a and 37a and an inner wall surface of a tip portion 33 (probe outer casing 317) form a hollow portion S2 having a circular disk shape. In this hollow portion S2, a sealing member 38 described above is provided. Similar to the above embodiment, the sealing member 38 is disposed at a position which does not interfere with at least an optical axis of illumination light emitted from the illumination fiber 311 (an axis extending from an end portion of an illumination fiber 311 in the longitudinal direction of the tip portion 33) and optical axes of light (return light) incident on a first light reception fiber 312, a second light reception fiber 313 and a third light reception fiber 314 (the axes extending from the end portion of each light reception fiber in the longitudinal direction of the tip portion 33).

Further, the glass member 36a is formed to have such quality of a material or the thickness that the glass member 36a is deformed when a predetermined load is applied. The sealing member 38 is broken following deformation of this glass member 36a, and releases the internal liquid Lq.

According to above-described Modified Example 1, the sealing member 38 is disposed in the hollow space S2 inside the glass window 35a, when a load of a predetermined magnitude or more is applied, the sealing member 38 is broken, and the liquid Lq sealed inside the sealing member 38 is released inside the hollow portion S2 covering at least the first light reception fiber 312, the second light reception fiber 313 and the third light reception fiber 314, and the transmittance is changed, so that it is possible to check whether or not optical performance of the measurement probe 3a is maintained to initial performance.

Further, according to present Modified Example 1, the glass members 36a and 37a having flat shapes are disposed without contacting each other, so that the glass member 36a is more likely to be deformed than a glass member 36 according to the above embodiment when an external load is applied. Consequently, it is possible to more precisely deform the glass member 36a when an external load is applied.

FIG. 7 is a view schematically illustrating a cross section obtained by cutting along the longitudinal direction a tip portion 33 of a measurement probe 3b according to Modified Example 2 of the embodiment of the present invention. As described in Modified Example 2, a glass window 35 may be arranged to project from a tip of a probe outer casing 317. By this means, a load is more preferentially applied to the glass window 35 when an external shock is applied, so that a transmittance change unit (a sealing member 38 and a liquid Lq) can change the transmittance when an external load is applied to a tip portion.

Incidentally, although, with the above embodiment, an optical element 34 and the glass window 35 have virtually circular disk shapes and are formed by using light transmissive glasses having a predetermined refractive index, the optical element 34 and the glass window 35 may be formed with lenses having a predetermined magnification power or reduction power or may be integrally formed.

Further, although the glass window 35 is formed by pasting glass members 36 and 37, the glass window 35 may be formed with one glass member as long as a hollow portion S1 can be formed.

Furthermore, although a black ink is used for a liquid Lq with the above present embodiment, any liquid is applicable as long as the liquid changes the transmittance with respect to a measurement probe 3 (light reception fiber). For example, when light incident on each light reception fiber is red, a blue ink which is an inverted color of red may be used, or even a transparent liquid is applicable as long as the transparent liquid can change the transmittance. A liquid is applicable as long as the liquid changes the transmittance of light having at least a measurement target wavelength.

Further, although the sealing member 38 which seals the liquid Lq and is broken when a load of a predetermined magnitude or more is applied is a transmittance change unit with the above present embodiment, a configuration without the sealing member 38 may be employed as long as the liquid Lq has a certain shape in a state where a load is not applied, and has viscosity to spread in the hollow portion S1 when a load of a predetermined magnitude or more is applied.

According to some embodiments, it is possible to check whether or not initial performance is maintained.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A measurement probe which is detachably connected to a living body optical measurement apparatus which optically measures a body tissue, the measurement probe comprising:
an illumination fiber which radiates illumination light on the body tissue;
a light reception fiber which receives return light of the illumination light reflected and/or scattered by the body tissue;
an optical element which abuts on the illumination fiber and the light reception fiber, and fixes a distance between the illumination fiber and the light reception fiber, and the body tissue;
an optical member which is provided at a tip of the measurement probe, and which comprises a hollow portion which forms a hollow space; and
a transmittance change unit which is provided in the hollow portion, and which changes a transmittance of light incident on an interior of the measurement probe in response to a load from an outside.

2. The measurement probe according to claim 1, wherein the transmittance change unit comprises:
a liquid which changes the transmittance of the light; and
a sealing member which seals the liquid and is broken due to a load of a predetermined magnitude or more.

3. The measurement probe according to claim 1, further comprising a probe outer casing which covers an outer peripheral surface of the illumination fiber, the light reception fiber, the optical element and the optical member,
wherein the optical member projects from a tip of the probe outer casing.

4. The measurement probe according to claim 2, further comprising a probe outer casing which covers an outer peripheral surface of the illumination fiber, the light reception fiber, the optical element and the optical member,
wherein the optical member projects from a tip of the probe outer casing.
